(19) United States
(12) Patent Application Publication
(10) Pub. No.: US 2008/0097219 A1
(43) Pub. Date: Apr. 24, 2008

(54) Thermographic Assessment Of Clostridial Toxin Applications

(75) Inventors: David H. Reser, Costa Mesa, CA (US); Joseph A. Francis, Aliso Viejo, CA (US); Lance E. Steward, Irvine, CA (US)

Correspondence Address: ALLERGAN, INC., 2525 DUPONT DRIVE, T2-7H, IRVINE, CA, 92612-1599, US

(73) Assignee: Allergan , Inc., Irvine, California (JP)

(21) Appl. No.: 11/571,197

(22) PCT Filed: Jul. 20, 2005
(86) PCT No.: PCT/US05/26290

§ 371 Date: Dec. 22, 2006

Related U.S. Application Data
(60) Provisional application No. 60/651,493, filed on Jul. 20, 2004.
(60) Provisional application No. 60/678,872, filed on May 6, 2005.

(30) Foreign Application Priority Data
Jul. 20, 2004 (US) 10894851

(51) Int. Cl. A61B 8/00; A61K 35/00; A61P 43/00

(52) U.S. Cl. 600/474; 424/780

(57) ABSTRACT

The present specification relates to methods for assessing the physiological activity of a target site being evaluated for potential administration of a Clostridial toxin, methods for administering a Clostridial toxin to a particular target site, methods for assessing the effect of an administration of a Clostridial toxin in a mammal and methods assessing the extent of dispersal of a Clostridial toxin from a target area to a non-target area in a mammal.

### CROSS REFERENCE TO RELATED APPLICATIONS

This is a national stage application under 35 U.S.C. § 371 of PCT application PCT/US2005/026290, filed on Jul. 20, 2005 which claims priority pursuant to 35 U.S.C. §119(e) to U.S. provisional patent application Ser. No. 60/651,493, filed Jul. 20, 2004, which was converted on Jul. 5, 2005 from U.S. nonprovisional patent application Ser. No. 10/894,851 filed Jul. 20, 2004 and pursuant to 35 U.S.C. §119(e) to U.S. provisional patent application Ser. No. 60/678,872 filed May 6, 2005, which are hereby incorporated by reference in their entirety.

All of the patents and publications cited in this application are hereby incorporated by reference in their entirety.

The ability of Clostridial toxins, such as, e.g., Botulinum neurotoxins (BoNTs), like, BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder. In addition, Clostridial toxins therapies are proposed for treating neuromuscular disorders, see e.g., Kei Roger Aoki et al., Method for Treating Neuromuscular Disorders and Conditions with Botulinum Toxin Types A and B, U.S. Pat. No. 6,872,397 (Mar. 29, 2005); Rhett M. Schiffman, Methods for Treating Uterine Disorders, U.S. Patent Publication No. 2004/0175399 (Sep. 9, 2004); Richard L. Barron, Methods for Treating Ulcers and Gastroesophageal Reflux Disease, U.S. Patent Publication No. 2004/0086531 (May. 7, 2004); and Kei Roger Aoki, et al., Method for Treating Dystonia with Botulinum Toxin C to G, U.S. Pat. No. 6,319,505 (Nov. 20, 2001); eye disorders, see e.g., Eric R. First, Methods and Compositions for Treating Eye Disorders, U.S. Patent Publication No. 2004/0234532 (Nov. 25, 2004); Kei Roger Aoki et al., Botulinum Toxin Treatment for Blepharospasm, U.S. Patent Publication No. 2004/0151740 (Aug. 5, 2004); and Kei Roger Aoki et al., Botulinum Toxin Treatment for Strabismus, U.S. Patent Publication No. 2004/0126396 (Jul. 1, 2004); pain, see e.g., Kei Roger Aoki et al., Pain Treatment by Peripheral Administration of a Neurotoxin, U.S. Pat. No. 6,869,610 (Mar. 22, 2005); Stephen Donovan, Clostridial Toxin Derivatives and Methods to Treat Pain, U.S. Pat. No. 6,641,820 (Nov. 4, 2003); Kei Roger Aoki, et al., Method for Treating Pain by Peripheral Administration of a Neurotoxin, U.S. Pat. No. 6,464,986 (Oct. 15, 2002); Kei Roger Aoki and Minglei Cui, Methods for Treating Pain, U.S. Pat. No. 6,113,915 (Sep. 5, 2000); Martin A. Voet, Methods for Treating Fibromyalgia, U.S. Pat. No. 6,623,742 (Sep. 23, 2003); Martin A. Voet, Botulinum Toxin Therapy for Fibromyalgia, U.S. Patent Publication No. 2004/0062776 (Apr. 1, 2004); and Kei Roger Aoki et al., Botulinum Toxin Therapy for Lower Back Pain, U.S. Patent Publication No. 2004/0037852 (Feb. 26, 2004); muscle injuries, see e.g., Gregory F. Brooks, Methods for Treating Muscle Injuries, U.S. Pat. No. 6,423,319 (Jul. 23, 2002); headache, see e.g., Martin Voet, Methods for Treating Sinus Headache, U.S. Pat. No. 6,838,434 (Jan. 4, 2005); Kei Roger Aoki et al., Methods for Treating Tension Headache, U.S. Pat. No. 6,776,992 (Aug. 17, 2004); and Kei Roger Aoki et al., Method for Treating Headache, U.S. Pat. No. 6,458,365 (Oct. 1, 2002); William J. Binder, Method for Reduction of Migraine Headache Pain, U.S. Pat. No. 5,714,469 (Feb. 3, 1998); cardiovascular diseases, see e.g., Gregory F. Brooks and Stephen Donovan, Methods for Treating Cardiovascular Diseases with Botulinum Toxin, U.S. Pat. No. 6,767,544 (Jul. 27, 2004); neurological disorders, see e.g., Stephen Donovan, Parkinson's Disease Treatment, U.S. Pat. No. 6,620,415 (Sep. 16, 2003); and Stephen Donovan, Method for Treating Parkinson's Disease with a Botulinum Toxin, U.S. Pat. No. 6,306,403 (Oct. 23, 2001); neuropsychiatric disorders, see e.g., Stephen Donovan, Botulinum Toxin Therapy for Neuropsychiatric Disorders, U.S. Patent Publication No. 2004/0180061 (Sep. 16, 2004); and Steven Donovan, Therapeutic Treatments for Neuropsychiatric Disorders, U.S. Patent Publication No. 2003/0211121 (Nov. 13, 2003); endocrine disorders, see e.g., Stephen Donovan, Method for Treating Endocrine Disorders, U.S. Pat. No. 6,827,931 (Dec. 7, 2004); Stephen Donovan, Method for Treating Thyroid Disorders with a Botulinum Toxin, U.S. Pat. No. 6,740,321 (May. 25, 2004); Kei Roger Aoki et al., Method for Treating a Cholinergic Influenced Sweat Gland, U.S. Pat. No. 6,683,049 (Jan. 27, 2004); Stephen Donovan, Neurotoxin Therapy for Diabetes, U.S. Pat. No. 6,416,765 (Jul. 9, 2002); Stephen Donovan, Methods for Treating Diabetes, U.S. Pat. No. 6,337,075 (Jan. 8, 2002); Stephen Donovan, Method for Treating a Pancreatic Disorder with a Neurotoxin, U.S. Pat. No. 6,261,572 (Jul. 17, 2001); Stephen Donovan, Methods for Treating Pancreatic Disorders, U.S. Pat. No. 6,143,306 (Nov. 7, 2000); cancers, see e.g., Stephen Donovan, Methods for Treating Bone Tumors, U.S. Pat. No. 6,565,870 (May 20, 2003); Stephen Donovan, Method for Treating Cancer with a Neurotoxin to Improve Patient Function, U.S. Pat. No. 6,368,605 (Apr. 9, 2002); Stephen Donovan, Method for Treating Cancer with a Neurotoxin, U.S. Pat. No. 6,139,845 (Oct. 31, 2000); and Mitchell F. Brin and Stephen Donovan, Methods for Treating Diverse Cancers, U.S. Patent Publication No. 2005/0031648 (Feb. 10, 2005); otic disorders, see e.g., Stephen Donovan, Neurotoxin Therapy for Inner Ear Disorders, U.S. Pat. No. 6,358,926 (Mar. 19, 2002); and Stephen Donovan, Method for Treating Otic Disorders, U.S. Pat. No. 6,265,379 (Jul. 24, 2001); autonomic disorders, see, e.g., Pankai J. Pasricha and Anthony N. Kaloo, Method for Treating Gastrointestinal Muscle Disorders and Other Smooth Muscle Dysfunction, U.S. Pat. No. 5,437,291 (Aug. 1, 1995); as well as other disorders, see e.g., William J. Binder, Method for Treatment of Skin Lesions Associated with Cutaneous Cell-proliferative Disorders, U.S. Pat. No. 5,670,484 (Sep. 23, 1997); Eric R. First, Application of Botulinum Toxin to the Management of Neurogenic Inflammatory Disorders, U.S. Pat. No. 6,063,768 (May 16, 2000); Marvin Schwartz and Brian J. Freund, Method to Reduce Hair Loss and Stimulate Hair Growth, U.S. Pat. No. 6,299,893 (Oct. 9, 2001); Jean D. A. Carruthers and Alastair Carruthers, Cosmetic Use of Botulinum Toxin for Treatment of Downturned Mouth, U.S. Pat. No. 6,358,917 (Mar. 19, 2002); Stephen Donovan, Use of a Clostridial Toxin to Reduce Appetite, U.S. Patent Publication No. 2004/40253274 (Dec. 16, 2004); and Howard I. Katz and Andrew M. Blumenfeld, Botulinum Toxin Dental Therapies and Procedures, U.S. Patent Publication No. 2004/0115139 (Jun. 17, 2004); Kei Roger Aoki, et al., Treatment of Neuromuscular Disorders and Conditions with Different Botulinum, U.S. Patent Publication No. 2002/0010138 (Jan. 24, 2002); and Kei Roger Aoki, et al., Use of Botulinum Toxins for Treating Various Disorders and Conditions and Associated Pain, U.S. Patent Publication No. 2004/0013692 (Jan. 22, 2004). In addition, the expected use of Clostridial toxins, such as, e.g., BoNTs, like., BoNT/A, BoNT/B, BoNT/C1, BoNT/D, BoNT/E, BoNT/F and BoNT/G, and TeNT, in therapeutic and cosmetic treatments of humans and other mammals is anticipated to expand to an ever widening range of diseases and aliments that can benefit from the properties of these toxins.

The growing clinical, therapeutic and cosmetic use of Clostridial toxins necessitates the pharmaceutical industry to use accurate assays for Clostridial toxin effects in order to, e.g., ensure accurate pharmaceutical formulations, monitor established quality control standards and evaluate medical treatment regimes. In addition, while Clostridial toxins are being used for a wide range of clinical, therapeutic and cosmetic interventions, current methods for assessing the degree of effect due to toxin administration are often rudimentary and subjective. For example, such methods often rely on observed clinical effects or visual inspection of muscle tone or activity or invasive techniques that measure neuronal activity. The present invention provides novel methods for determining more precisely the administration sites of a Clostridial toxin to a mammal, as well as, methods for assessing the effects of a Clostridial toxin administration in a mammal. These and related advantages are useful for various clinical, therapeutic and cosmetic applications, such as, e.g. the treatment of neuromuscular disorders, neuropathic disorders, eye disorders, pain, muscle injuries, headache, cardiovascular diseases, neuropsychiatric disorders, endocrine disorders, cancers, otic disorders, hyperkinetic facial lines, as well as, other disorders where a Clostridial toxin administration to a mammal can produce a beneficial effect.

### DETAILED DESCRIPTION OF THE INVENTION

Thermal imaging, or thermography, visualizes the amount of thermal energy being emitted from a surface. Thermography has been applied in various fields of medicine, veterinary medicine, pharmacy, and dentistry as a valuable diagnostic tool that can potentially differentiate between a diseased and a non-diseased state. These applications take advantage of the fact that surface temperature of the body reflects the activity of underlying physiological processes and their effects on blood circulation. For example, the surface temperature distribution of the skin in a healthy mammalian body exhibits a bilateral symmetry, whereas perturbations in a physiological activity underlying a particular disease or disorder can be associated with an abnormal thermal pattern of the surface, i.e., the loss of bilateral symmetry in the thermal pattern. Thus, a physiological dysfunction can be revealed by either an increase or a decrease in the amount of thermal energy being emitted from the body surface. Current medical applications of thermographic systems include, e.g., detection of blood flow as applied in, e.g., coronary artery bypass surgery, microsurgery, wound healing, peripheral vascular disorders and deep vein thrombosis; staging and analysis of burn trauma; inflammatory diseases; reproductive problems; cancer risk assessment and prognosis; diabetes; pain; neurological problems; neuro-musculoskeletal diseases; and autonomic nervous diseases. Thermal imaging is, therefore, an effective technique for examining both normal and abnormal physiological changes and responses.

The present invention provides, in part, novel methods for assessing the physiological activity of a target site being evaluated for potential administration of a Clostridial toxin. These novel methods take advantage of the fact that abnormal physiological activity underling regions that could benefit from an administration of a Clostridial toxin will emit thermal energy that is different from the thermal energy emitted by an area not requiring such a Clostridial toxin treatment. In addition, the present invention provides, in part, novel methods for assessing the effect of an in vivo administration of a Clostridial toxin in a mammal using thermography. These novel methods rely on the difference in thermal energy emitted from an area affected by a Clostridial toxin as compared to the thermal energy emitted from an area unaffected by the toxin. Such differences can be useful, e.g., in assessing which particular area or areas in a mammal should be administered a Clostridial toxin; in administering a Clostridial toxin to a particular area or areas; in assessing the extent of Clostridial toxin administration and whether additional toxin should be administered; and in assessing the extent of dispersal of a Clostridial toxin from a target area to a non-target area in a mammal.

Thus, aspects of the present invention provide methods of assessing a physiological activity of a target site for administration of a Clostridial toxin to a mammal, the method comprising the step of recording a thermal image from a surface of the target site in the mammal prior to a Clostridial toxin administration.

Other aspects provide methods of administering a Clostridial toxin to a target site in a mammal, the method comprising the steps of recording a thermal image from a surface of the target site in the mammal before administration of a Clostridial toxin; and administering the Clostridial toxin to the target site.

Other aspects provide methods of assessing the effect of a Clostridial toxin to a target site in a mammal, the method comprising the steps of a) recording a thermal image from a surface of the target site in the mammal before administration of the Clostridial toxin; b) recording a second thermal image from the surface of the target site in the mammal after administration of the Clostridial toxin; and c) comparing the thermal image of step (a) to the thermal image of step (b).

Other aspects provide methods of assessing dispersal of a Clostridial toxin from a target site to a non-target site in a mammal, the method comprising the steps of a) recording a thermal image from a surface of the target site in the mammal and from a surface of the non-target site in the mammal before administration of the Clostridial toxin; b) recording a second thermal image from the surface of the target site in the mammal and from the surface of the non-target site in the mammal after administration of the Clostridial toxin; and c) comparing the thermal image of the target site and the thermal image of the non-target site of step (a) to the thermal image of the target site and the thermal image of the non-target site of step (b).

Aspects of the present invention provide methods of assessing a physiological activity of a target site for administration of a Clostridial toxin to a mammal, the method comprising the step of recording a thermal image from a surface of the target site in the mammal prior to a Clostridial toxin administration. As used herein, the term “mammal” includes, but not limited to, rodents, rabbits, porcines, bovines, equines, non-human primates and humans. As a non-limiting example, a target site for administering a Clostridial toxin can be identified by assessing a physiological activity of a target site in a mammal using a thermal imaging system.

Aspects of the present invention provide, in part, assessing a physiological activity. As used herein, the term “physiological activity” means any process that generates heat resulting in the emission of thermal energy from a surface in a mammal. As used herein, the term “surface” means any body area that can emit thermal energy, such as, e.g., a skin surface or a surface of an exposed internal body part like a muscle, organ or gland. Many physiological activities can generate heat, such as, e.g., a metabolic activity, a neuronal activity, a hemodynamic activity and a muscle activity. Metabolic activities includes, without limitation, an anabolic activity and a catabolic activity. Neuronal activities includes, without limitation, an autonomic neuronal activity; a motor neuronal activity; and a sensory neuronal activity, involving, e.g., a nociceptive stimuli and a non-nociceptive stimuli, like, a chemical stimuli, a thermal stimuli and a mechanical stimuli. As heat is generated by physiological activity in a mammal, it is distributed throughout the body by the circulating blood. Since the interior body temperature of a mammal is usually higher than the surrounding ambient temperature, a temperature gradient produces heat flow from the inside of the body's core to the body's surface. The extent of this temperature gradient is regulated by the blood flow to the surface. As a non-limiting example, vasodilation of the capillaries at the skin surface increases blood flow, which in turn, increases the conduction of heat, thereby increasing the amount of thermal energy emitted from the skin surface. Vasoconstriction of the capillaries in the skin decrease blood flow, which in turn, decrease the conduction of heat, thereby decreasing the amount of thermal energy emitted from the skin surface.

It is envisioned that any disease or disorder benefiting from a Clostridial toxin treatment which exhibits a disrupted physiological activity that results in the emission of thermal energy that is different than a non-disease or non-disorder state can be assessed using methods disclosed in the present specification. Non-limiting examples of such diseases and disorders include, e.g., neuromuscular disorders, neuropathic disorders, movement disorders, eye disorders, pain, muscle injuries, headache, cardiovascular diseases, neuropsychiatric disorders, endocrine disorders, cancers, otic disorders and myokinesis disorders. As a non-limiting example, a muscle undergoing hyperkinesia or muscle spasm, such as, e.g., focal dystonias like blepharospasm, oromandubular dystonia, spasmodic dystonia, cervical dystonia, task-specific dystonias, segmental dystonias, general dystonia, myoclonus, tics and tremors, exhibits physiological activity, such as, e.g., a motor neuronal activity, different than a muscle not experiencing hyperkinesia or muscle spasm. This difference in physiological activity results in a different amount of thermal energy being emitted from the muscle undergoing hyperkinesia or muscle spasm as compared to the muscle not experiencing hyperkinesia or muscle spasm. A thermal image will reveal the muscle undergoing hyperkinesia or muscle spasm, and thus, identify a region that can potentially be treated by administering a Clostridial toxin. As another non-limiting example, abnormal control in axillary sweat gland function resulting in sweating beyond what is physiological necessary to maintain normal thermoregulation, such as, e.g., primary hyperhidrosis, secondary hyperhydrosis and idiopathic hyperhydrosis exhibiting a physiological activity, such as, e.g., an autonomic neuronal activity, different than a normally functioning sweat gland. A thermal image will reveal the sweat gland undergoing abnormal sweating, and thus, identify a region that can potentially be treated by administering a Clostridial toxin. As another non-limiting example, a body region experiencing pain, such as, e.g., inflammatory pain and neuropathic pain, exhibits physiological activity, such as, e.g., a sensory neuronal activity, different than a body region not experiencing pain. This difference in physiological activity results in a different amount of thermal energy being emitted from the region experiencing pain as compared to the region not experiencing pain. A thermal image will reveal the body region experiencing pain, and thus, identify a region that can potentially be treated by administering a Clostridial toxin.

Thus, in an embodiment, a target site is assessed for a physiological activity by recording a thermal image of a surface in a mammal. In another embodiment, a target area is assessed for metabolic activity by recording a thermal image of a surface in a mammal. In aspects of this embodiment, a target area is assessed for, e.g., anabolic activity by recording a thermal image of a surface or catabolic activity by recording a thermal image of a surface. In another embodiment, a target area is assessed for neuronal activity by recording a thermal image of a surface in a mammal. In aspects of this embodiment, a target area is assessed for, e.g., autonomic neuronal activity by recording a thermal image of a surface, motor neuronal activity by recording a thermal image of a surface or sensory neuronal activity by recording a thermal image of a surface. In further aspects of this embodiment, a target area is assessed for, e.g., sensory neuronal activity involving a nociceptin stimulus by recording a thermal image of a surface or sensory neuronal activity involving a non-nociceptin stimulus by recording a thermal image of a surface. In other aspects of this embodiment, a target area is assessed for, e.g., a sensory neuronal activity involving a chemical stimulus by recording a thermal image of a surface, a sensory neuronal activity involving a thermal stimulus by recording a thermal image of a surface or a sensory neuronal activity involving a mechanical stimulus by recording a thermal image of a surface. In yet another embodiment, a target area is assessed for hemodynamic activity by recording a thermal image of a surface in a mammal. In a further embodiment, a target area is assessed for muscle activity by recording a thermal image of a surface in a mammal.

In another embodiment, a target site is assessed for a physiological activity by recording a thermal image of a surface. In an aspect of this embodiment, a target site is assessed for a physiological activity by recording a thermal image of a skin surface. In another aspect of this embodiment, a target site is assessed for a physiological activity by recording a thermal image of a muscle surface. In yet another aspect of this embodiment, a target site is assessed for a physiological activity by recording a thermal image of an organ surface. In still another aspect of this embodiment, a target site is assessed for a physiological activity by recording a thermal image of a gland surface.

Aspects of the present invention provide, in part, assessing a target site. As used herein, the term “target site” means a particular area of a mammalian body for which administration of a Clostridial toxin is being considered or is desired. Non-limiting examples of a target site can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, stomach, pancreas, colon, uterus, thyroid gland, parathyroid gland, prostate gland and sweat glands.

Thus, in an embodiment a target site is assessed for administration of a Clostridial toxin. In aspects of this embodiment, a target site being assessed for administration of a Clostridial toxin can be, e.g., a muscle, a skin region, an organ or a gland. In further aspects of this embodiment, a target muscle site being assessed for administration of a Clostridial toxin can be, e.g., a skeletal muscle, a smooth muscle or a cardiac muscle. In yet further aspects of this embodiment, target skin site being assessed for administration of a Clostridial toxin can be, e.g., epidermal skin, dermal skin, subdermal skin and cutaneous skin or subcutaneous skin. In still further aspects of this embodiment, a target organ site being assessed for administration of a Clostridial toxin can be, e.g., bladder, stomach, pancreas, colon, uterus, thyroid gland, parathyroid gland, prostate gland or sweat gland.

Aspects of the present invention provide, in part, recording a thermal image of a surface. As used herein, the term “recording a thermal image” means detecting the thermal energy emitted from a target site and/or a non-target site. It is envisioned that any and all thermographic systems that can record a thermal image can be used, such as, e.g., liquid crystal thermography (LCT), infrared thermography (IRT), microwave thermography (MWT) and Computerized thermal imaging (CTI). In general, thermographic systems, use an infrared sensor to convert thermal energy into electric signals thereby producing a thermal image. The thermal image can be generated by means of either an optical scanning system or a pyroelectric vidicon television tube. A video monitor or the like can be used to display the image. Non-limiting examples of thermographic systems include, e.g., Albert F. Kutas and Demetro U. Tokaruk, Scanning Thermography, U.S. Pat. No. 3,862,423 (Jan. 21, 1975); Robert P. Hunt and Richard H. Winkler, Infrared Imaging System, U.S. Pat. No. 3,909,521 (Sep. 30, 1975); Victor J. Anselmo and Terrence H. Reilly, Medical Diagnosis System and Method With Multispectral Imaging, U.S. Pat. No. 4,170,987 (Oct. 16, 1979); Peter T. Walsall and James R. Vincent, Method for Identifying the Presence of Abnormal Tissue, U.S. Pat. No. 4,428,382 (Jan. 31, 1984); Frank K. Leung, Apparatus for Thermographic Examinations, U.S. Pat. No. 4,548,212 ((Oct. 22, 1985); Toshio Murotani, Infrared Imaging Device, U.S. Pat. No. 5,034,794 (Jul. 23, 1991); Akio Tanaka, Infrared Imaging Device and Infrared Imaging System Using Same, U.S. Pat. No. 5,594,248 (Jan. 14, 1997); Zhong Qi Liu and Chen Wang, Method and Apparatus for Thermal Radiation Imaging, U.S. Pat. No. 6,023,637 (Feb. 8, 2000); Liang-Chien Chu and Chih-Chi Chang, Infrared 3D Scanning System, U.S. Pat. No. 6,442,419 (Aug. 27, 2002); and Tae-woo Kim et al., Non-Invasive Apparatus for Measuring A Temperature of A Living Body and Method Therefor, U.S. Pat. No. 6,773,159 (Aug. 10, 2004). In addition, thermographic systems are commercially available, such as, e.g., Teletherm infrared imager (Ashwin Systems International, Inc., Tampa, Fla.); Meditherm med2000™ (Mediterm, Beaufort, N.C.); Thermal Image Processor™ System (Computerized Thermal Imaging, Inc., Ogden, Utah) and TSA ImagIR (Seahorse Bioscience Inc., North Billerica, Mass.).

The skin temperature varies dynamically and continuously depending on the thermoregulatory state of the mammal. During a resting condition, the body and ambient temperature are allowed to equilibrate to some extent which causes the skin capillaries to vasoconstrict in an effort to conserve thermal energy and maintain the core temperature of the body. During a non-resting condition, a stress is applied to the body which causes the skin capillaries to vasodilate in an effort to release thermal energy and reduce the core temperature of the body. Non-resting conditions can be induced by, without limitation, a thermal stress, such as, e.g., cooling or heating, mechanical stress, such as, e.g., vibration or physical exertion, or chemical stress, such as, e.g., vasodilators or vasoconstrictors. Thus, a resting condition will reflect a certain thermoregulatory state whereas a non-resting condition will reflect a different thermoregulatory state from that of the resting condition. It is understood that a resting condition may not always produce a maximal difference in the thermal energy emitted. Thus, in order to exacerbate the difference in thermal energy being emitted from a region exhibiting an abnormal physiological activity as compared to a region exhibiting normal physiological activity, a thermal image of a mammal may be taken under non-resting conditions.

Thus, in one embodiment, a thermal image recording can be done during a resting condition. In another embodiment, a plurality of thermal image recordings can be done during a resting condition. In yet another embodiment, a thermal image recording can be done during a non-resting condition. In yet another embodiment, a plurality of thermal image recordings can be done during a non-resting condition. As a non-limiting example of a resting condition, the target area is exposed to the environment, e.g., by removing any clothing or shaving away fur, and the mammal takes a comfortable, relaxing position in a climate controlled room held at approximately 18-22±1° C. for a period of approximately 10-30 minutes. As a non-limiting example of a non-resting condition, the target area is exposed to the environment, e.g., by removing any clothing or shaving away fur, and the mammal undergoes physical exertion, such as, e.g., running in place, on a read mill, on an exercise wheel, in a climate controlled room held at approximately 18-22±1° C. for a period of approximately 5-30 minutes.

Thermal imaging can record thermal energy over the entire body surface of a mammal to detect systemic thermal variation or this technique can record thermal energy of a discrete body surface to detect localized thermal variation. Thus, in one embodiment, recording of a thermal image can be done over an entire body surface of a mammal to detect systemic thermal variation. In another embodiment, recording of a thermal image can be done at a discrete body surface to detect localized thermal variation.

Other aspects of the present invention provide methods of administering a Clostridial toxin to a target site in a mammal, the method comprising the steps of recording a thermal image from a surface of the target site in the mammal before administration of a Clostridial toxin; and administering the Clostridial toxin to the target site. As a non-limiting example, examining a thermal image will identify a target site, thereby provide information regarding where to administer a Clostridial toxin in a mammal.

Aspects of the present invention provide, in part, administering a Clostridial toxin to a target site. Non-limiting examples of a target site that is administered a Clostridial toxin can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, stomach, pancreas, colon, uterus, thyroid gland, parathyroid gland, prostate gland and sweat glands.

Thus, in an embodiment a target site is administered a Clostridial toxin. In aspects of this embodiment, a target site that is administered a Clostridial toxin can be, e.g., a muscle, a skin region, an organ or a gland. In further aspects of this embodiment, a target muscle site being administered a Clostridial toxin can be, e.g., a skeletal muscle, a smooth muscle or a cardiac muscle. In yet further aspects of this embodiment, target skin site being administered a Clostridial toxin can be, e.g., epidermal skin, dermal skin, subdermal skin and cutaneous skin or subcutaneous skin. In still further aspects of this embodiment, a target organ or gland site being administered a Clostridial toxin can be, e.g., bladder, stomach, pancreas, colon, uterus, thyroid gland, parathyroid gland, prostate gland or sweat gland.

Aspects of the present invention provide, in part, recording a thermal image before administration of a Clostridial toxin. As used herein, the term “before” means any length of time prior to the actual administration of a Clostridial toxin to a mammal. In one embodiment, the recording of a thermal image occurs before administration of a Clostridial toxin. Aspects of this embodiment include recording a thermal image, e.g., at least one minute before administration of a Clostridial toxin, at least 5 minutes before administration of a Clostridial toxin, at least 15 minutes before administration of a Clostridial toxin, at least 30 minutes before administration of a Clostridial toxin, at least 45 minutes before administration of a Clostridial toxin or at least 60 minutes before administration of a Clostridial toxin. Other aspects of this embodiment include recording a thermal image, e.g., at least one hour before administration of a Clostridial toxin, at least two hours before administration of a Clostridial toxin, at least four hours before administration of a Clostridial toxin, at least eight hours before administration of a Clostridial toxin, at least 12 hours before administration of a Clostridial toxin or at least 24 hours before administration of a Clostridial toxin. Further aspects of this embodiment include recording a thermal image, e.g., at least one day before administration of a Clostridial toxin, at least two days before administration of a Clostridial toxin, at least four days before administration of a Clostridial toxin, at least eight days before administration of a Clostridial toxin, at least 15 days before administration of a Clostridial toxin or at least 30 days before administration of a Clostridial toxin.

Additional aspects of this embodiment include recording a thermal image, e.g., at most one minute before administration of a Clostridial toxin, at most 5 minutes before administration of a Clostridial toxin, at most 15 minutes before administration of a Clostridial toxin, at most 30 minutes before administration of a Clostridial toxin, at most 45 minutes before administration of a Clostridial toxin or at most 60 minutes before administration of a Clostridial toxin. Still other aspects of this embodiment include recording a thermal image, e.g., at most one hour before administration of a Clostridial toxin, at most two hours before administration of a Clostridial toxin, at most four hours before administration of a Clostridial toxin, at most eight hours before administration of a Clostridial toxin, at most 12 hours before administration of a Clostridial toxin or at most 24 hours before administration of a Clostridial toxin. Still further aspects of this embodiment include recording a thermal image, e.g., at most one day before administration of a Clostridial toxin, at most two days before administration of a Clostridial toxin, at most four days before administration of a Clostridial toxin, at most eight days before administration of a Clostridial toxin, at most 15 days before administration of a Clostridial toxin or at most 30 days before administration of a Clostridial toxin.

Aspects of the present invention provide, in part, administration of a Clostridial toxin. As used herein, the term “administration” means any means that provides a Clostridial toxin to a target tissue that potentially results in a clinically, therapeutically, cosmetically or experimentally beneficial result. Administration can be local or systemic. Local administration results in significantly more Clostridial toxin being delivered to a specific location as compared to the entire body of the subject, whereas, systemic administration results in delivery of a Clostridial toxin to essentially the entire body of the subject. Administration of a Clostridial toxin can be by any means including, without limitation, orally in any acceptable form, such as, e.g., tablet, liquid, capsule, powder, or the like; topically in any acceptable form, such as, e.g., patch, drops, creams, gels or ointments; by injection, in any acceptable form, such as, e.g., intravenous, intraperitoneal, intramuscular, subcutaneous, parenteral or epidural; and by implant, such as, e.g., subcutaneous pump, intrathecal pump or other bioerodible or non-bioerodible implanted extended release device or formulation. In general administration of a Clostridial toxin to a mammal can depend on, e.g., the type and location of the disorder, the toxin or other molecule to be included in the composition, and the history, risk factors and symptoms of the mammal.

Thus, in one embodiment, a Clostridial toxin is administered to a target site. In aspects of this embodiment, a Clostridial toxin is administered orally to a target site, a Clostridial toxin is administered topically to a target site, a Clostridial toxin is injected to a target site or a Clostridial toxin is implanted in a target site.

Aspects of the present invention provide, in part, administration of a Clostridial toxin. Clostridial toxins are found in many species belonging to the genus Clostridium, including, without limitation, C. botulinum, C. tetani, C. baratii and C. butyricum. Seven antigenically-distinct serotypes of Botulinum toxins (BoNTs) have been identified by investigating botulism outbreaks in man (BoNT/A, /B, /E and /F), animals (BoNT/C1 and /D), or isolated from soil (BoNT/G). It is recognized by those of skill in the art that within each type of Clostridial toxin there can be subtypes that differ somewhat in their amino acid sequence, and also in the nucleic acids encoding these proteins. For example, BoNT/A subtypes include, e.g., BoNT/A1, BoNT/A2, BoNT/A3 and BoNT/A4; BoNT/B subtypes include, e.g., BoNT/B1, BoNT/B2, BoNT/B bivalent and BoNT/B nonproteolytic; BoNT/C1 subtypes include, e.g., BoNT/C1-1 and BoNT/C1-2; and BoNT/E subtypes include BoNT/E1 , BoNT/E2 and BoNT/E3. Tetanus toxin (TeNT) appears to be produced by a uniform group of C. tetani, while C. baratii and C. butyricum, also produce toxins similar to BoNT/F and BoNT/E, respectively. Clostridial toxins commercially available as pharmaceutical compositions include, BoNT/A preparations, such as, e.g., BOTOX® (Allergan, Inc., Irvine, Calif.), Dyspor®/Reloxin®, (Beaufour Ipsen, Porton Down, England), Linurase® (Prollenium, Inc., Ontario, Canada), Neuronox® (Medy-Tox, Inc., Ochang-myeon, South Korea) BTX-A (Lanzhou Institute Biological Products, China) and Xeomin® (Merz Pharmaceuticals, GmbH., Frankfurt, Germany); and BoNT/B preparations, such as, e.g., MyoBloc™/NeuroBloc™ (Elan Pharmaceuticals, San Francisco, Calif.). Furthermore, Clostridial toxins include active fragments, chimeras, and other recombinant derivatives useful for clinical, therapeutic and cosmetic applications. Such toxins are disclosed in, e.g., Clifford C. Shone et al., Recombinant Toxin Fragments, U.S. Pat. No. 6,461,617 (Oct. 8, 2002); Keith A. Foster et al., Clostridial Toxin Derivatives Able To Modify Peripheral Sensory Afferent Functions, U.S. Pat. No. 6,395,513 (May 28, 2002); Wei-Jin Lin et al., Neurotoxins with Enhanced Target Specificity, US 2002/0137886 (Sep. 26, 2002); Keith A. Foster et al., Inhibition of Secretion from Non-neural Cells, US 2003/0180289 (Sep. 25, 2003); J. Oliver Dolly et al., Activatable Recombinant Neurotoxins, WO 2001/014570 (Mar. 1, 2001); Clifford C. Shone et al., Recombinant Toxin Fragments, WO 2004/024909 (Mar. 25, 2004); and Keith A. Foster et al., Re-targeted Toxin Conjugates, WO 2005/023309 (Mar. 17, 2005).

Thus, in an embodiment, a Clostridial toxin is administered to a target site. In aspects of this embodiment, a Botulinum toxin is administered to a target site, a Tetanus toxin is administered to a target site, a C. baratii toxin is administered to a target site or a C. butyricum toxin is administered to a target site. In other aspects of this embodiment, a Clostridial toxin administered to a target site can be, e.g., a BoNT/A, a BoNT/B, a BoNT/C1, a BoNT/D, a BoNT/E, a BoNT/F or a BoNT/G. In still other aspects of this embodiment, a Clostridial toxin administered to a target site can be, e.g., a BOTOX® preparation, a Dyspor®/Reloxin® preparation, a Linurase® preparation, a Neuronox® preparation, a BTX-A preparation, a Xeomin® preparation or a MyoBloc™/NeuroBloc™ preparation. In yet other aspects of this embodiment, a Clostridial toxin administered to a target site can be, e.g., a recombinant Clostridial toxin, an active fragment of a Clostridial toxin, a Clostridial toxin derivative or a chimeric Clostridial toxin.

The specific dosage administered to a mammal depends on several factors, including, without limitation, the size and type of the target site to be treated, the type and severity of the disease or disorder to be treated, the weight and age of the mammal, the responsiveness of the mammal to a treatment and the particular commercial preparation of the Clostridial toxin. For example, 18 U/kg total body weight of a BOTOX® preparation, with a per use maximum dose of 400 units are administered to patients suffering from spasticity. Appropriate administration is readily determined by one of ordinary skill in the art according to the factors discussed above. As a non-limiting example, approximately 75-125 units of BOTOX® per intramuscular injection (multiple muscles) are administered to a patient undergoing treatment for cervical dystonia. As another non-limiting example, approximately 5-10 units of BOTOX® per intramuscular injection (5 units injected intramuscularly into the procerus muscle and 10 units injected intramuscularly into each corrugator supercilii muscle) is administered to a patient undergoing treatment for glabellar lines (brow furrows). As another non-limiting example, approximately 30-80 units of BOTOX® is administered to a patient undergoing treatment for constipation by intrasphincter injection of the puborectalis muscle. As yet another non-limiting example, approximately 1-5 units per muscle of intramuscularly injected BOTOX® is administered to a patient undergoing treatment for blepharospasm by injecting the lateral pre-tarsal orbicularis oculi muscle of the upper lid and the lateral pre-tarsal orbicularis oculi of the lower lid. As yet another non-limiting example, approximately 1-5 units of BOTOX® is administered to a patient undergoing treatment for strabismus, the dose of toxin intramuscular injected of the extraocular muscles depending upon both the size of the muscle to be injected and the extent of muscle paralysis desired (i.e. amount of diopter correction desired). As still another non-limiting example, upper limb spasticity following stroke is treated by intramuscular injections of BOTOX® into five different upper limb flexor muscles, as follows: (a) flexor digitorum profundus: 7.5 units to 30 units; (b) flexor digitorum sublimus: 7.5 units to 30 units; (c) flexor carpi ulnaris: 10 units to 40 units; (d) flexor carpi radialis: 15 units to 60 units; (e) biceps brachii: 50 units to 200 units. Each of the five indicated muscles has been injected at the same treatment session, so that the patient receives from 90 units to 360 units of upper limb flexor muscle BOTOX® by intramuscular injection at each treatment session. As still another non-limiting example, approximately 25 units of BOTOX® is administered by pericranial injected (injected symmetrically into glabellar, frontalis and temporalis muscles) to a patient undergoing treatment for migraine.

Other aspects provide methods of assessing the effect of a Clostridial toxin on a target site in a mammal, the method comprising the steps of a) recording a thermal image from a surface of the target site in the mammal before administration of a Clostridial toxin; b) recording a second thermal image from the surface of the target site in the mammal after administration of a Clostridial toxin; and c) comparing the thermal image of step (a) to the thermal image of step (b). As a non-limiting example, a particular toxin parameter, such as, e.g., the efficacy of the toxin, the stability of a toxin or the effectiveness of the toxin, can be determined by assessing the effect of a Clostridial toxin administration in a mammal using a thermal imaging system. As another non-limiting example, a particular treatment parameter, such as, e.g., safety margins of a treatment, degree of success of the treatment or the identification of the location of a subsequent toxin administration, can be determined by assessing the effect of a Clostridial toxin administration in a mammal using a thermal imaging system. As another non-limiting example, a particular intra-target parameter, such as, e.g., the distribution of toxin effect within one muscle, skin region, organ or gland, can be determined by assessing the effect of a Clostridial toxin administration in a mammal using a thermal imaging system. As yet another non-limiting example, the effective lethal dose of a Clostridial toxin formulation can be determined by assessing the effect of a Clostridial toxin administration in a mammal using a thermal imaging system. As yet another non-limiting example, immunoresistance to a Clostridial toxin can be determined by assessing the effect of a Clostridial toxin administration in a mammal using a thermal imaging system.

Aspects of the present invention provide, in part, assessing the effect of a Clostridial toxin. As used herein, the term “effect” means a change in a physiological activity that is a direct or indirect result of a Clostridial toxin activity, such as, e.g., disruption of a SNARE-mediated process. Non-limiting examples of a Clostridial toxin effect can include, e.g., inhibiting the release of a neuronal molecule, such as, e.g., a neurotransmitter, a neuromodulator, a neuropeptide or a neurohormone; inhibiting the release of a non-neuronal molecule, such as, e.g., a growth factor, a cytokine, a hormone, an enzyme or a lipid; inhibiting an activity of, e.g., a muscle, a skin region, an organ or a gland. In vitro studies indicated that Clostridial toxins inhibit potassium cation induced release of both acetylcholine and norepinephrine from primary cell cultures of brainstem tissue; inhibit the evoked release of both glycine and glutamate in primary cultures of spinal cord neurons; and inhibit the release of acetylcholine, dopamine, norepinephrine, CGRP, substance P and glutamate in brain synaptosome preparations. The neuronal molecules listed above, mediate a wide range of neuronal activities including, without limitation, autonomic neuronal activity; motor neuronal activity; and sensory neuronal activity. As a non-limiting example, a therapeutically effective amount of BOTOX® administered into the underlying facial muscles inhibits the release of the neurotransmitter acetylcholine at the neuromuscular junction thereby relieving hyperkinetic facial lines of the forehead. As another non-limiting example, a therapeutically effective amount of BOTOX® administered into the sweat glands inhibits the release of neurotransmitters from the autonomic neurons controlling sweat release, thereby reducing the symptoms of hyperhidrosis. As yet another non-limiting example, a therapeutically effective amount of BOTOX® administered into the skin reduces the pain response evoked by sensory neurons and local vasomotor reaction of the surrounding blood vessels.

Thus, in an embodiment a target site is assessed for a Clostridial toxin effect. In aspects of this embodiment, a Clostridial toxin effect is assessed by a change in, e.g., a release of a neuronal molecule, a release of a non-neuronal molecule or an activity of a muscle, a skin region, an organ or a gland. In further aspects of this embodiment, a Clostridial toxin effect is assessed by a change in a release of a neuronal molecule, such as, e.g., a neurotransmitter, a neuromodulator, a neuropeptide or a neurohormone. In yet further aspects of this embodiment, a Clostridial toxin effect is assessed by a change in a release of a non-neuronal molecule, such as, e.g., a growth factor, a cytokine, a hormone, an enzyme or a lipid.

Aspects of the present invention provide, in part, assessing the effect of a Clostridial toxin by recording a thermal image from a surface of a target site. Non-limiting examples of a surface that a thermal image can be recorded can include, e.g., a skin surface, a muscle surface, an organ surface or a gland surface. Non-limiting examples of a target site being assessed for a Clostridial toxin effect can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, stomach, pancreas, colon, uterus, thyroid gland, parathyroid gland, prostate gland and sweat glands.

Thus, in an embodiment a target site is assessed for a Clostridial toxin effect. In aspects of this embodiment, a target site being assessed for a Clostridial toxin effect can be, e.g., a muscle, a skin region, an organ or a gland. In further aspects of this embodiment, a target muscle site being assessed for a Clostridial toxin effect can be, e.g., a skeletal muscle, a smooth muscle or a cardiac muscle. In yet further aspects of this embodiment, target skin site being assessed for a Clostridial toxin effect can be, e.g., epidermal skin, dermal skin, subdermal skin and cutaneous skin or subcutaneous skin. In still further aspects of this embodiment, a target organ or gland site being assessed for a Clostridial toxin effect can be, e.g., bladder, stomach, pancreas, colon, uterus, thyroid gland, parathyroid gland, prostate gland or sweat gland.

In another embodiment, assessing the effect of a Clostridial toxin by recording a thermal image from a surface of a target site. In an aspect of this embodiment, a Clostridial toxin effect is assessed by recording a thermal image of a skin surface of a target site. In another aspect of this embodiment, a Clostridial toxin effect is assessed by recording a thermal image of a muscle surface of a target site. In yet another aspect of this embodiment, a Clostridial toxin effect is assessed by recording a thermal image of an organ surface of a target site. In still another aspect of this embodiment, a Clostridial toxin effect is assessed by recording a thermal image of a gland surface of a target site.

Aspects of the present invention provide, in part, recording a thermal image after administration of a Clostridial toxin. As used herein, the term “after” means any length of time following the actual administration of a Clostridial toxin to a mammal. Thus aspects of this embodiment include recording a thermal image, e.g., at least one minute after administration of a Clostridial toxin, at least 5 minutes after administration of a Clostridial toxin, at least 15 minutes after administration of a Clostridial toxin, at least 30 minutes after administration of a Clostridial toxin, at least 45 minutes after administration of a Clostridial toxin or at least 60 minutes after administration of a Clostridial toxin. Other aspects of this embodiment include recording a thermal image, e.g., at least one hour after administration of a Clostridial toxin, at least two hours after administration of a Clostridial toxin, at least four hours after administration of a Clostridial toxin, at least eight hours after administration of a Clostridial toxin, at least 12 hours after administration of a Clostridial toxin or at least 24 hours after administration of a Clostridial toxin. Further aspects of this embodiment include recording a thermal image, e.g., at least one day after administration of a Clostridial toxin, at least two days after administration of a Clostridial toxin, at least four days after administration of a Clostridial toxin, at least eight days after administration of a Clostridial toxin, at least 15 days after administration of a Clostridial toxin or at least 30 days after administration of a Clostridial toxin.

Additional aspects of this embodiment include recording a thermal image, e.g., at most one minute after administration of a Clostridial toxin, at most 5 minutes after administration of a Clostridial toxin, at most 15 minutes after administration of a Clostridial toxin, at most 30 minutes after administration of a Clostridial toxin, at most 45 minutes after administration of a Clostridial toxin or at most 60 minutes after administration of a Clostridial toxin. Still other aspects of this embodiment include recording a thermal image, e.g., at most one hour after administration of a Clostridial toxin, at most two hours after administration of a Clostridial toxin, at most four hours after administration of a Clostridial toxin, at most eight hours after administration of a Clostridial toxin, at most 12 hours after administration of a Clostridial toxin or at most 24 hours after administration of a Clostridial toxin. Still further aspects of this embodiment include recording a thermal image, e.g., at most one day after administration of a Clostridial toxin, at most two days after administration of a Clostridial toxin, at most four days after administration of a Clostridial toxin, at most eight days after administration of a Clostridial toxin, at most 15 days after administration of a Clostridial toxin or at most 30 days after administration of a Clostridial toxin.

Aspects of the present invention provide, in part, comparing a thermal image with another thermal image. As used herein, the term “comparing” means detecting a thermal variation between two or more different regions on a single thermal image or detecting a thermal variation of the same region from two or more thermal images. Thus, in aspects of this embodiment, comparing a thermal image with another thermal image can involve, e.g., comparing two or more target sites, comparing two or more non-target sites, comparing a target site to a non-target site, comparing a target site before administration of a Clostridial toxin to the same target site after administration of a Clostridial toxin, comparing a non-target site before administration of a Clostridial toxin to a target site to the same non-target site after administration of a Clostridial toxin to that target site, comparing two or more target site before administration of a Clostridial toxin to the same two or more target site after administration of a Clostridial toxin or comparing two or more non-target sites before administration of a Clostridial toxin to a target site to the same two or more non-target sites after administration of a Clostridial toxin to that target site.

Comparing a thermal image with another thermal image can be qualitative or quantitative. Qualitative comparisons can involve visual assessment of images by one skilled in the art to detect thermal variations, such as, e.g., hot or cold spot thermal variations and symmetrical or asymmetrical thermal variations. Quantitative comparisons can involve automated or semi-automated computerized assessment of images to detect thermal variations. As non-limiting examples, BTHERM and CTHERM are open systems for capturing, storing, retrieving and manipulating sequences of thermal images, see, e.g., Bryan F Jones and Peter Plassmann, Digital Infrared Thermal Imaging of Human Skin, 21(6). IEEE Eng. Med. Biol. Mag. 41-48 (2002). Thus in aspects of this embodiment, comparing a thermal image with another thermal image involves detecting a thermal variation of, e.g., at least 0.025° C., at least 0.05° C., at least 0.075° C., at least 0.1° C., at least 0.25° C., at least 0.5° C., at least 0.75° C., at least 1.0° C., at least 2.0° C. or at least 5.0° C. In other aspects of this embodiment, comparing a thermal image with another thermal image involves detecting a thermal variation of, e.g., at most 0.025° C., at most 0.05° C., at most 0.075° C., at most 0.1° C., at most 0.25° C., at most 0.5° C., at most 0.75° C., at most 1.0° C., at most 2.0° C. or at most 5.0° C.

The magnitude of thermal energy variation detected by comparing a thermal image with another thermal image is proportional to the degree of Clostridial toxin effect. As a non-limiting example, detecting a thermal increase of 1° C. in a target site, obtained by comparing thermal images of that target site before and after toxin administration, is indicative of a greater Clostridial toxin effect than detecting a thermal increase of 0.1° C. in a target site, obtained by comparing thermal images of that target site before and after toxin administration. Likewise, detecting a thermal decrease of 1° C. in a target site, obtained by comparing thermal images of that target site before and after toxin administration, is indicative of a greater Clostridial toxin effect than detecting a thermal decrease of 0.1° C. in a target site, obtained by comparing thermal images of that target site before and after toxin administration.

Other aspects provide methods of assessing dispersal of a Clostridial toxin from a target site to a non-target site in a mammal, the method comprising the steps of a) recording a first thermal image from a surface of the target site in the mammal and from a surface of the non-target site in the mammal before administration of the Clostridial toxin; b) recording a second thermal image from the surface of the target site in the mammal and from the surface of the non-target site in the mammal after administration of the Clostridial toxin; and c) comparing the thermal image of the target site and the thermal image of the non-target site of step (a) to the thermal image of the target site and the thermal image of the non-target site of step (b). As a non-limiting example, local diffusion of a Clostridial toxin in a mammal can be determined by assessing the dispersal of a Clostridial toxin from a target site to a non-target site using a thermal imaging system. As another non-limiting example, systemic diffusion of a Clostridial toxin in a mammal can be determined by assessing the dispersal of a Clostridial toxin from a target site to a non-target site using a thermal imaging system.

Aspects of the present invention provide, in part, assessing the dispersal of a Clostridial toxin. As used herein, the term “dispersal” means any mode of passive or active transportation of a Clostridial toxin from a target site to a non-target site, including, without limitation, movement by diffusion, movement by passive transport, movement by active transport, movement by the circulatory system, movement by the lymphatic system and movement by retrograde transport.

Aspects of the present invention provide, in part, assessing the dispersal of a Clostridial toxin by recording a thermal image from a surface of a target site. Non-limiting examples of a surface that a thermal image can be recorded can include, e.g., a skin surface, a muscle surface, an organ surface or a gland surface. Non-limiting examples of a target site being assessed for dispersal of a Clostridial toxin can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, stomach, pancreas, colon, uterus, thyroid gland, parathyroid gland, prostate gland and sweat glands. assessing dispersal of a Clostridial toxin from a target site

Thus, in an embodiment, the dispersal of a Clostridial toxin is assessed by recording a thermal image from a surface of a target site. In aspects of this embodiment, a target site being assessed for dispersal of a Clostridial toxin can be, e.g., a muscle, a skin region, an organ or a gland. In further aspects of this embodiment, a target muscle site being assessed for dispersal of a Clostridial toxin can be, e.g., a skeletal muscle, a smooth muscle or a cardiac muscle. In yet further aspects of this embodiment, target skin site being assessed for dispersal of a Clostridial toxin can be, e.g., epidermal skin, dermal skin, subdermal skin and cutaneous skin or subcutaneous skin. In still further aspects of this embodiment, a target organ or gland site being assessed for dispersal of a Clostridial toxin can be, e.g., bladder, stomach, pancreas, colon, uterus, thyroid gland, parathyroid gland, prostate gland or sweat gland.

In another embodiment, the dispersal of a Clostridial toxin is assessed by recording a thermal image from a surface of a target site. In an aspect of this embodiment, the dispersal of a Clostridial toxin is assessed by recording a thermal image of a skin surface of a target site. In another aspect of this embodiment, the dispersal of a Clostridial toxin is assessed by recording a thermal image of a muscle surface of a target site. In yet another aspect of this embodiment, the dispersal of a Clostridial toxin is assessed by recording a thermal image of an organ surface of a target site. In still another aspect of this embodiment, the dispersal of a Clostridial toxin is assessed by recording a thermal image of a gland surface of a target site.

Aspects of the present invention provide, in part, assessing the dispersal of a Clostridial toxin by recording a thermal image from a surface of a non-target site. As used herein, the term “non-target site” means a particular area of a mammalian body for which administration of a Clostridial toxin is not being considered or is undesired. Non-limiting examples of a non-target site being assessed for dispersal of a Clostridial toxin can include muscle, such as, e.g., skeletal or striated muscle, smooth muscle like visceral muscle and vascular muscle and cardiac muscle; skin, such as, e.g., epidermal skin, dermal skin, subdermal skin and cutaneous skin and subcutaneous skin; and organs, such as, e.g., bladder, stomach, pancreas, colon, uterus, thyroid gland, parathyroid gland, prostate gland and sweat glands. Non-limiting examples of a surface that a thermal image can be recorded can include, e.g., a skin surface, a muscle surface, an organ surface or a gland surface. Generally, administration of a Clostridial toxin is well tolerated. However, the administered toxin may diffuse to areas other than the target site, namely a non-target site, particularly when high toxin doses are administered. For example, a patient administered a therapeutically effective amount MyoBloc™/NeuroBloc™ into the neck muscles for torticollis may develop dysphagia because of dispersal of the toxin into the oropharynx.

Thus, in an embodiment a non-target site is assessed for dispersal of a Clostridial toxin from a target site. In aspects of this embodiment, a non-target site assessed for dispersal of a Clostridial toxin can be, e.g., a muscle, a skin region, an organ or a gland. In further aspects of this embodiment, a non-target muscle site being assessed for dispersal of a Clostridial toxin can be, e.g., a skeletal muscle, a smooth muscle or a cardiac muscle. In yet further aspects of this embodiment, non-target skin site being assessed for dispersal of a Clostridial toxin can be, e.g., epidermal skin, dermal skin, subdermal skin and cutaneous skin or subcutaneous skin. In still further aspects of this embodiment, an non-target organ or gland site being assessed for dispersal of a Clostridial toxin can be, e.g., bladder, stomach, pancreas, colon, uterus, thyroid gland, parathyroid gland, prostate gland or sweat gland.

In another embodiment, the dispersal of a Clostridial toxin is assessed by recording a thermal image from a surface of a non-target site. In an aspect of this embodiment, the dispersal of a Clostridial toxin is assessed by recording a thermal image of a skin surface of a non-target site. In another aspect of this embodiment, the dispersal of a Clostridial toxin is assessed by recording a thermal image of a muscle surface of a non-target site. In yet another aspect of this embodiment, the dispersal of a Clostridial toxin is assessed by recording a thermal image of an organ surface of a non-target site. In still another aspect of this embodiment, the dispersal of a Clostridial toxin is assessed by recording a thermal image of a gland surface of a non-target site.

It is envisioned that dispersal of toxin from a target site to a non-target site can be detected at any and all distances according to the methods disclosed in the present specification, with the proviso that the dispersal distance is within the range of detection sensitivity of the thermographic system being used. The dispersal distance of a Clostridial toxin can be evaluated locally, e.g., by assessing the toxin's effect in the non-target sites immediately surrounding the target site, or systemically, e.g., by assessing the toxin's effect in the non-target sites not nearby the target site, such as, e.g., a region proximal to the target site, a region distal to the target site, a region ipsilateral to the target site or a region contralateral to the target site. The dispersal distance of a Clostridial toxin can be evaluated within the same muscle, skin region, organ or gland, or the dispersal distance of a Clostridial toxin can be evaluated between two or more different muscles, skin regions, organs or glands.

Thus, in one embodiment, the dispersal of a Clostridial toxin can be detected in non-target sites immediately surrounding the target site. In another embodiment, the dispersal of a Clostridial toxin can be detected in non-target sites not nearby the target site. In yet another embodiment, the dispersal of a Clostridial toxin can be detected locally. In yet another embodiment, the dispersal of a Clostridial toxin can be detected systemically. In aspects of this embodiment, the dispersal of a Clostridial toxin can be detected in a non-target site at a distance of, e.g., at most 0.1 cm from the target site, at most 0.5 cm from the target site, at most 1.0 cm from the target site, at most 5.0 cm from the target site, at most 10 cm from the target site, at most 50 cm from the target site, at most 100 cm from the target site and at most 150 cm from the target site. In other aspects of this embodiment, the dispersal of a Clostridial toxin can be detected in a non-target site at a distance of, e.g., at least 0.1 cm from the target site, at least 0.5 cm from the target site, at least 1.0 cm from the target site, at least 5.0 cm from the target site, at least 10 cm from the target site, at least 50 cm from the target site, at least 100 cm from the target site and at least 150 cm from the target site.

Comparing a thermal image with another thermal image can be qualitative or quantitative. Qualitative comparisons can involve visual assessment of images by one skilled in the art to detect thermal variations, such as, e.g., hot or cold spot thermal variations and symmetrical or asymmetrical thermal variations. Quantitative comparisons can involve automated or semi-automated computerized assessment of images to detect thermal variations. As non-limiting examples, BTHERM and CTHERM are open systems for capturing, storing, retrieving and manipulating sequences of thermal images, see, e.g., Bryan F Jones and Peter Plassmann, Digital Infrared Thermal Imaging of Human Skin, 21(6). IEEE Eng. Med. Biol. Mag. 41-48 (2002). Thus in aspects of this embodiment, comparing a thermal image with another thermal image involves detecting a thermal variation of, e.g., at least 0.025° C., at least 0.05° C., at least 0.075° C., at least 0.1° C., at least 0.25 ° C., at least 0. 5° C., at least 0.75° C., at least 1.0° C., at least 2.0° C. or at least 5.0° C. In other aspects of this embodiment, comparing a thermal image with another thermal image involves detecting a thermal variation of, e.g., at most 0.025° C., at most 0.05° C., at most 0.075° C., at most 0.1° C., at most 0.25° C., at most 0. 5° C., at most 0.75° C., at most 1.0° C., at most 2.0° C. or at most 5.0° C.

The magnitude of thermal energy variation detected by comparing a thermal image with another thermal image is proportional to the degree of Clostridial toxin dispersal. As a non-limiting example, detecting a thermal increase of 1° C. in a non-target site, obtained by comparing thermal images of that non-target site before and after toxin administration, is indicative of greater Clostridial toxin dispersal than a detecting a thermal increase of 0.1° C. in a non-target site, obtained by comparing thermal images of that non-target site before and after toxin administration. Likewise, detecting a thermal decrease of 1° C. in a non-target site, obtained by comparing thermal images of that non-target site before and after toxin administration, is indicative of a greater Clostridial toxin dispersal than detecting a thermal decrease of 0.1° C. in a non-target site, obtained by comparing thermal images of that non-target site before and after toxin administration.

### EXAMPLES

The following non-limiting examples are provided for illustrative purposes only in order to facilitate a more complete understanding of disclosed embodiments and are in no way intended to limit any of the embodiments disclosed in the present invention.

### Example 1

### Assessing a Physiological Activity of a Target Site for a Clostridial Toxin Administration

This example illustrates how examining a thermal image can identify a target site for administering a Clostridial toxin.

A 44 year old male patient suffers from intense pain due to a task-specific dystonia affecting the palm and fingers of the right hand. To determine the location of the dystonic areas as well as to assess whether a Clostridial toxin administration would be appropriate for treating this affliction, the physician employs thermal imaging technique to assess the physiological activity of the man's palm and fingers of the right hand.

The male patient is prepared for thermal imaging under resting conditions. The male patient is prepared for thermal imaging under resting conditions. This is done by asking the patient to disrobe the affected area and letting the patient lie down in a supine position in a climate controlled room held at 20±1° C. for a period of approximately 25 minutes. The scanner unit is positioned at a distance of approximately 20 cm perpendicular to the affected hand, thereby allowing maximum coverage of the target site. A thermal image of the hand is then taken and the digital image is stored on a computer hard drive. One thermographic imaging system that may be used in accordance with aspects of the present invention is the Agema Thermovision 900 series (AGEMA Infrared Systems AB, Danderyd, Sweden). The scanner is a long-wave, cryogenically cooled system utilizing a mercury cadmium telluride detector with a spectral response of 8-12 μm and a sensitivity of 0.1° C. at 30° C. This window of 8-12 μm coincides with the region of maximal skin emission of 8-10 μm. The scanner is controlled by a dedicated system controller which runs software specifically for thermal image analysis. After visual examination of the thermal image, the physician determines that three muscle groups show a dramatically increase in thermal energy being emitted from the affected hand as compared to the male patient's unaffected left hand and with thermal images of hands that are taken from other patients unaffected by task-specific dystonia. The physician recommends administering a Clostridial toxin to the muscle groups showing increased thermal energy emittance to alleviate the task-specific dystonia.

### Example 2

### Administering a Clostridial Toxin to a Target Site

This example illustrates how examining a thermal image will provide information regarding where to administer a Clostridial toxin to a target site.

A 38 year old female patient presents with a severe case of axillary hyperhidrosis. The treating physician recommends administering a botulinum toxin type A to the affected areas of hyperhidrosis. To determine which sweat glands to treat, the physician employs a thermal imaging technique to assess the physiological activity of the axillary areas undergoing excessive sweating.

The female patient is prepared for thermal imaging under resting conditions. This is done by asking the patient to disrobe the affected area and letting the patient lie down in a supine position in a climate controlled room held at 20±1° C. for a period of approximately 15 minutes. Once the patient becomes acclimated to the environment, she is then seated in a dental chair. The scanner unit is positioned at a distance of approximately 50 cm perpendicular to the affected axillary area in order to achieve maximum coverage of the target site. A thermal image of the area is then taken using, e.g., the thermographic imaging system described in Example 1, and the digital image is stored on a computer hard drive. Computer analysis of the thermal image reveals five target sites encompassing an 8×15 cm² region that exhibit a statistically significant increase in the thermal energy being emitted from the affected areas of hyperhidrosis in the female patient as compared to other patients unaffected by axillary hyperhidrosis.

The physician then proceeds to administer a Clostridial toxin to the areas showing increased thermal energy emittance to alleviate the excessive sweating. Based on the thermal image, the target sites are mapped within the 8×15 cm² region. Crystal ice particle coated with Botulinum toxin type A are loaded into a needleless injector. The projection pressure is set so that the drug particles may be delivered to the dermis layer of the skin. Also, the amount of the drug particle is loaded so that approximately 20 units to approximately 60 units of botulinum toxin type A is delivered to the five target sites within the 8×15 cm² region.

### Example 3

### Assessing the Effect of a Clostridial Toxin Administration

This example illustrates how comparing a thermal image of a target site before and after the administration of a Clostridial toxin will provide information regarding the degree of a Clostridial toxin effect on that target site.

A 53 year old female patient suffers from intense pain due to a temporomandibular joint dysfunction. The treating physician recommends administering 10 units of botulinum toxin type A to her masseter muscles to alleviate the pain. To determine the muscle sites to treat, the physician employs a thermal imaging technique to assess the physiological activity of the area undergoing intense pain.

The female patient is prepared for thermal imaging under resting conditions. This is done by asking the patient to disrobe the affected area and letting the patient lie down in a supine position in a climate controlled room held at 20±1° C. for a period of approximately 15 minutes. In addition, all facial cosmetics are removed and the skin surface allowed to air dry. Hair is held back off the face with hair clips and a reflective marker is placed on the skin overlying the anterior edge of the masseter muscle. Once the patient becomes acclimated to the environment, she is then seated in a dental chair. The scanner unit is positioned at a distance of approximately 30 cm perpendicular to the affected temporomandibular joint area in order to achieve maximum coverage of the target site. Thermal images of both sides of the face are then taken using, e.g., the thermographic imaging system described in Example 1, and the digital images are stored on a computer hard drive. Computer analysis of the thermal image reveals that the masseter muscle on both sides of the face exhibit a statistically significant increase in the thermal energy being emitted from the affected experiencing pain in the female patient as compared to other patients unaffected by temporomandibular joint dysfunction.

The physician then proceeds to administer a Clostridial toxin to the areas showing increased thermal energy emittance to alleviate the pain. Based on the thermal image, the target sites within the masseter muscle are mapped and the physician administers approximately 10 units of botulinum toxin type A to the target sites within the masseter muscles on each side of the patient's face of the patient. The patient is discharged and is asked to return for a second scan in 24 hours. Further, to prevent the unwanted dispersal of botulinum toxin into the adjacent muscles, the patient is instructed to not massage the administration site, and is advised to not reapply her makeup in the office.

The next day, the female patient returns as is prepared for thermal imaging under resting conditions as described above. Thermal images of both sides of the face are then taken using, e.g., the thermographic imaging system described in Example 1, and the digital images are stored on a computer hard drive. The thermographic system includes software that calculates the temperature differences between the first and the second thermal image. The alignment and subtraction of images is undertaken by superimposing two reference markers on each of the images of interest. For greater accuracy, surface reference markers of a highly reflective nature should be placed over recognized anatomical sites prior to the functional test. These markers allow for greater accuracy in the overlay procedure and therefore a more accurate result after pixel subtraction. Comparison of the two images indicate that the muscles administered botulinum toxin show a decrease in temperature which approximates the temperature exhibited by masseter muscles from patients unaffected by temporomandibular joint dysfunction. This comparison also shows that muscles not administered botulinum toxin show a temperature change of approximately 0° C. Upon analysis of these thermal images, the physician determines that additional administration of botulinum toxin is not warranted.

### Example 4

### Assessing the Dispersal of a Clostridial Toxin Administration

This example illustrates how comparing a thermal image of a target site before and after the administration of a Clostridial toxin will provide information regarding the degree of a Clostridial toxin effect on a target site and any possible dispersal of the toxin away from the target site to a non-target site.

A 33 year old male patient suffers from intense pain due to a muscle spasm in his left calf. The treating physician recommends administering 10 units of botulinum toxin type A to the calf muscle to alleviate the pain. To make sure that the administered botulinum toxin does not diffuse to unintended muscles, the physician employs a thermal imaging technique to assess the physiological activity of the area undergoing intense pain.

The male patient is prepared for thermal imaging under resting conditions. This is done by asking the patient to disrobe the affected area and letting the patient lie down in a supine position in a climate controlled room held at 20±1° C. for a period of approximately 25 minutes. The scanner unit is positioned at a distance of approximately 50 cm perpendicular to the affected leg in order to achieve maximum coverage of both the target and non-target sites. A thermal image of both the affected left calf and the unaffected right calf areas are then taken using, e.g., the thermographic imaging system described in Example 1, and the digital image is stored on a computer hard drive. Computer analysis of the thermal image reveals three target sites that exhibit a statistically significant increase in the thermal energy being emitted from the spasmodic calf area of the male patient as compared to the unaffected right calf area of the male patient as well as other patients not experiencing muscle spasms in the calf.

The physician then proceeds to administer a Clostridial toxin to the areas showing increased thermal energy emittance to alleviate the muscle spasm and associated pain. Based on the thermal image, the target sites within the calf muscles are mapped and the physician administers approximately 10 units of botulinum toxin type A to the target sites within the muscles of the left calf. The patient is discharged and is asked to return for a second scan in 24 hours. Further, to prevent the unwanted dispersal of botulinum toxin into the adjacent muscles, the patient is instructed to not massage the target site and avoid exertion on the day of treatment.

The next day, the male patient returns and is prepared for thermal imaging under resting conditions as described above. Thermal images of the left and right calves are then taken using, e.g., the thermographic imaging system described in Example 1, and the digital images are stored on a computer hard drive. Analysis of temperature differences between the first and the second thermal image are performed, e.g., as described in Example 3. Comparison of the two images indicate that most of the calf muscles administered botulinum toxin show a decrease in temperature which approximates the temperature exhibited by the unaffected right calf muscles from the patient. However, a small region from one of the identified target sites still emits an increased thermal energy, indicating an additional toxin administration is required. In addition, examination of the non-target sites reveal that these sites do not show a change in thermal energy emittence, indicating that the toxin did not diffuse into these non-target sites. Therefore, upon analysis of these thermal images, the physician determines that additional administration of botulinum toxin should be administered in the remaining target site showing a difference in thermal energy relative to the unaffected right calf muscle.

### Example 5

### Assessing the Effective Threshold Toxic Dose of a Clostridial Toxin Administration Using a Systemic Assay

This example illustrates how the effective threshold dose of a Clostridial toxin formulation can be determined by assessing the effect of a Clostridial toxin administration in a mammal using a thermal imaging system.

Currently, the effective lethal dose of a Clostridial toxin is determined using an in vivo assay that measures animal lethality, such as, e.g., the mouse lethality assay (MLA or LD₅₀ assay). The standard LD₅₀ assay evaluates the dose-dependent lethality of toxin preparations. However, the high doses of a Clostridial toxin necessary to achieve lethality also result in a systemic hypothermic response in the animal due to the disruption of many physiological processes that effect thermalregulation. This induced hypothermic response, due to the systemic responses to a Clostridial toxin administration, can be used as a readout of a toxin effect. In addition, Clostridial toxin-mediated changes in the physiological state of a mammal occur well before the onset of lethality. Thus, the detectable thermal energy changes resulting from the milder toxicity of lower non-lethal doses of a toxin can be used as an assay endpoint to determine the threshold systemic effects of a toxin rather than the lethal effects of the toxin. Therefore determining the effective threshold dose of a Clostridial toxin using a thermal imaging assay will greatly reduce the pain and suffering of the animals.

To determine the effective threshold dose of a Clostridial toxin formulation, the effect of a Clostridial toxin administration is assessed using a thermal imaging assay. Mice are prepared for thermal imaging under resting conditions. Mice are then lightly anesthetized with Isoflurane and a thermal image of the ventral thorax region from each mouse is acquired using a TSA ImagIR System (Seahorse Bioscience, North Billerica, Mass.) and the digital image is stored on a computer hard drive. Various doses of a BoNT/A formulation are then administered to the mice following recovery from anesthesia. In a typical assay, a BoNT/A stock solution is used to generate dose dilutions over a dose range of 30-60 U/kg (e.g. 30 U/kg, 36 U/kg, 42 U/kg, 48 U/kg, 54 U/kg, and 60 U/kg), with dilutions made in vehicle (0.5% BSA/saline solution). Dosing is based on the median weight per dose group, with mouse weights ranging from 17 grams to 30 grams, where the weight range of any single dose group of mice is no greater than +/−2 grams, and dose groups consist of 10 mice each. Mice are injected intraperitoneally with either vehicle (control) or the specific toxin dose dilution, delivered via a 27 gauge needle, and each mouse receives a dose volume based on individual weight, such that the desired dose (in U/kg) is delivered in a volume of 10 mL/kg.

The next day, each mouse is prepared for thermal imaging under non-resting conditions as described above. A whole body thermal image of each mouse is then taken using, e.g., the thermographic imaging system described above, and the digital images are stored on a computer hard drive. Analysis of temperature differences between the first and the second thermal image for each dose are performed, e.g., as described in Example 3 and a dose response curve is derived from nonlinear regression analysis of these data, establishing a thermographic dose-response measure of non-lethal toxicity. The dose that results in 50% of the mice exhibiting a statistically significant decrease in the thermal energy being emitted after administration of the BoNT/A preparation as compared to control animals, e.g., the thermal images of the same mice before administration of the BoNT/A preparation or different mice administered a saline control (a normothermic vehicle control group), is the effective threshold dose (TD₅₀).

### Example 6

### Assessing the Effective Threshold Pharmacological Dose of a Clostridial Toxin Administration Using a Local Target Site Assay

This example illustrates how the effective threshold dose of a Clostridial toxin formulation can be determined by assessing the effect of a Clostridial toxin administration in a mammal using a thermal imaging system.

The effective pharmacological dose of a Clostridial toxin is determined using an in vivo assay that measures muscle paralysis, such as, e.g., the Digit Abduction Score (DAS) assay or the Gastrocnemius Paralysis Assay (GPA). Muscle paralysis results in a decrease in the heat output by an exercised muscle due to failure in muscle contraction and the generation of heat. This decrease in induced thermal output following exercise (hypothermia) can be used as a readout of a Clostridial toxin effect. The doses used in pharmacological studies are non-toxic and non lethal and should only elicit paralytic responses in the muscle of injection (e.g. gastrocnemius) or in adjacent muscles as a measure of local intermuscular diffusion (e.g. tibialis anterior, extensor digitorum longus, quadriceps).

To determine the effective pharmacological dose of a Clostridial toxin formulation, the effect of a Clostridial toxin administration is assessed using a thermal imaging assay. Mice are prepared for thermal imaging under non-resting conditions by physical stimulation using a treadmill (15 degree incline@ 5 meters/minute) for 10 minutes. Mice are then lightly anesthetized with Isoflurane and thermal images are acquired of the skin (fur shaved) overlying the right (ipsilateral) and left (contralateral) gastroxcnemius muscles (fur shaved) of each mouse using a TSA ImagIR System (Seahorse Bioscience, North Billerica, Mass.) and the digital image is stored on a computer hard drive. Various doses of a BoNT/A formulation are administered to the mice by intramuscular injection in the tail (five mice/dose). In a typical assay, a BoNT/A stock solution is used to generate dose dilutions over a dose range of 1-60 U/kg, with dilutions made in vehicle (0.5% BSA/saline solution). Dosing is based on the median weight per dose group, with mouse weights ranging from 17 grams to 30 grams, where the weight range of any single dose group of mice is no greater than +/−4 grams, and dose groups consist of 6 mice each. The injection volume is 5 or 10 μL, delivered via a 30 gauge needle using a Hamilton syringe affixed with a ratcheting, volume-adjustable dispenser. BoNT/A solution is injected into the distal portion of the medial head of the right gastrocnemeus muscle, using the posterior medial malleolar groove as a needle guide. Progressive paralysis is then evaluated daily for four days to capture the peak paralytic effects (typically between three to four days post-injection of BoNT/A). Prior to collection of thermal images, mice are similarly exercised on the treadmill to stimulate muscle activity, followed by anesthesia and thermography. Images from test groups are compared (qualitatively and quantitatively) to images from the vehicle control group (normothermic). The degree of hypothermia (net difference from vehicle control) is calculated for each dose administered and a ED₅₀ (the dose producing 50% paralysis) is derived from nonlinear regression analysis of these data, establishing a thermographic dose-response measure of muscle paralysis.

### Example 7

### Assessing Immunoresistance to a Clostridial Toxin Administration

This example illustrates how immunoresistance to a Clostridial toxin can be determined by assessing the effect of a Clostridial toxin administration in a mammal using a thermal imaging system.

Immunoresistance to a Clostridial toxin in a mammal is usually determined using an in vivo assay that measures animal lethality, such as, e.g., the mouse protection assay (MPA). The current standard MPA evaluates the degree of protection conferred by anti-Clostridial toxin-neutralizing antibodies against a lethal challenge dose of a toxin. However, the high doses of a Clostridial toxin necessary to achieve lethality also result in a systemic hypothermic response in the animal due to the disruption of many physiological processes that effect thermoregulation. This induced hypothermic response, due to the systemic responses to a Clostridial toxin administration, can be used as a readout of a toxin effect. In addition, Clostridial toxin-mediated changes in the physiological state of a mammal occur well before the onset of lethality. Thus, the detectable thermal energy changes resulting from the milder toxicity of lower non-lethal doses of a toxin can be used to infer the presence of toxin-neutralizing antibodies since the presence of toxin-neutralizing antibodies will effectively lower the challenge toxin dose (when combined), producing a graded toxicity response to the otherwise lethal challenge dose. Therefore determining the presence of anti-Clostridial toxin-neutralizing antibodies using a thermal imaging assay will greatly reduce the pain and suffering of the animals.

To determine the immunoresistance of a cervical dystonia patient, the effect of a Clostridial toxin administration is assessed using a thermal imaging assay. First, the maximum toxic dose (LD₉₉) of a BoNT/A preparation is determine, e.g., as described in Example 5, except that the various doses of a BoNT/A formulation are administered to the mice by intravenous injection in the tail. Second, mice are prepared for thermal imaging under resting conditions. Mice are then lightly anesthetized with Isoflurane and a thermal image of the ventral thorax region from each mouse is acquired using a TSA ImagIR System (Seahorse Bioscience, North Billerica, Mass.) and the digital image is stored on a computer hard drive. A blood sample from each patient is processed to obtain the serum. A 100 μL aliquot of serum from each patient is mixed with a 100 μL aliquot of a LD₉₉ dose of a BoNT/A preparation and incubated for 60 minutes in a 22° C. water bath. Toxin dosing is based on the median weight per dose group, with mouse weights ranging from 17 grams to 30 grams, where the weight range of any single dose group of mice is no greater than +/−2 grams. The negative control is vehicle (0.5% BSA/saline solution) and the positive control is a hyperimmune rabbit serum containing a high titer of toxin-neutralizing antibodies. These test samples are then administered to the mice by intravenous injection in the tail (five mice/dose).

The next day, each mouse is prepared for thermal imaging under resting conditions as described above. A whole body thermal image of each mouse is then taken using, e.g., the thermographic imaging system described above, and the digital images are stored on a computer hard drive. Analysis of temperature differences between the first and the second thermal image for each test sample is performed, e.g., as described in Example 3. In addition, test mice are compared (qualitatively and quantitatively) to images from the positive control group (full protection; normothermic) and the negative control group (no protection; maximally hypothermic). Mice that do not exhibit a statistically significant increase in the thermal energy being emitted after administration of the test sample, i.e., protected from BoNT/A toxicity, as compared to control animals, e.g., the thermal images of negative control mice administered the LD₉₉ of the BoNT/A preparation, indicate that the patient has developed an immunoresistant response to the BoNT/A preparation (i.e. that toxin-neutralizing antibodies are present in the patient serum).

Although the invention has been described with reference to the examples provided above, it should be understood that various modifications can be made without departing from the spirit of the invention. Accordingly, the invention is limited only by the following claims.

What is claimed:

1. A method of assessing a physiological activity of a target site for administration of a Clostridial toxin to a mammal, the method comprising the step of recording a thermal image from a surface of the target site in the mammal prior to a Clostridial toxin administration.

2. The method according to claim 1, wherein the recording is taken under resting conditions.

3. The method according to claim 1, wherein the recording is taken under non-resting conditions.

4. The method according to claim 1, wherein the surface comprises a muscle surface, a skin surface, an organ surface or a gland surface.

5. The method according to claim 1, wherein the target site comprises a muscle, a skin region, an organ or a gland.

6. The method according to claim 1, wherein the mammal consists of a rodent, a rabbit, a porcine, a bovine, an equine, a non-human primate or a human.

7. A method of administering a Clostridial toxin to a target site in a mammal, the method comprising the steps of:
a. recording a thermal image from a surface of the target site in the mammal prior to a Clostridial toxin administration; and
b. administering the Clostridial toxin to the target site.

8. The method according to claim 7, wherein the recording is taken under resting conditions.

9. The method according to claim 7, wherein the recording is taken under non-resting conditions.

10. The method according to claim 7, wherein the surface comprises a muscle surface, a skin surface, an organ surface or a gland surface.

11. The method according to claim 7, wherein the target site comprises a muscle, a skin region, an organ or a gland.

12. The method according to claim 7, wherein the mammal consists of a rodent, a rabbit, a porcine, a bovine, an equine, a non-human primate or a human.

13. The method according to claim 7, wherein administering the Clostridial toxin is by injection.

14. A method of assessing an effect of a Clostridial toxin on a target site in a mammal, the method comprising the steps of:
a. recording a first thermal image from a surface of the target site in the mammal prior to administration of the Clostridial toxin;
b. recording a second thermal image from the surface of the target site in the mammal after the administration of the Clostridial toxin; and
c. comparing the thermal image of step (a) to the thermal image of step (b).

15. The method according to claim 14, wherein the first thermal image recording is taken under resting conditions.

16. The method according to claim 14, wherein the first thermal image recording is taken under non-resting conditions.

17. The method according to claim 14, wherein the second thermal image recording is taken under resting conditions.

18. The method according to claim 14, wherein the second thermal image recording is taken under non-resting conditions.

19. The method according to claim 14, wherein the surface comprises a muscle surface, a skin surface, an organ surface or a gland surface.

20. The method according to claim 14, wherein the target site comprises a muscle, a skin region, an organ or a gland.

21. The method according to claim 14, wherein the mammal consists of a rodent, a rabbit, a porcine, a bovine, an equine, a non-human primate or a human.

22. The method according to claim 14, wherein administering the Clostridial toxin is by injection.

23. The method according to claim 14, wherein the comparison of step (c) is qualitative.

24. The method according to claim 14, wherein the comparison of step (c) is quantitative.

25. A method of assessing dispersal of a Clostridial toxin from a target site to a non-target site in a mammal, the method comprising the steps of:
a. recording a first thermal image from a surface of the target site in the mammal and from a surface of the non-target site of the mammal prior to administration of the Clostridial toxin;
b. recording a second thermal image from the surface of the target site in the mammal and from the surface of the non-target site of the mammal after the administration of the Clostridial toxin; and
c. comparing the thermal image of the target site and the thermal image of the non-target site of step (a) to the thermal image of the target site and the thermal image of the non-target site of step (b).

26. The method according to claim 25, wherein the first thermal image recording is taken under resting conditions.

27. The method according to claim 25, wherein the first thermal image recording is taken under non-resting conditions.

28. The method according to claim 25, wherein the second thermal image recording is taken under resting conditions.

29. The method according to claim 25, wherein the second thermal image recording is taken under non-resting conditions.

30. The method according to claim 25, wherein the target site surface comprises a muscle surface, a skin surface, an organ surface or a gland surface.

31. The method according to claim 25, wherein the target site comprises a muscle, a skin region, an organ or a gland.

32. The method according to claim 25, wherein the non-target site surface comprises a muscle surface, a skin surface, an organ surface or a gland surface.

33. The method according to claim 25, wherein the non-target site comprises a muscle, a skin region, an organ or a gland.

34. The method according to claim 25, wherein the mammal consists of a rodent, a rabbit, a porcine, a bovine, an equine, a non-human primate or a human.

35. The method according to claim 25, wherein administering the Clostridial toxin is by injection.

36. The method according to claim 25, wherein the comparison of step (c) is qualitative.

37. The method according to claim 25, wherein the comparison of step (c) is quantitative.
